(19) United States
(12) Patent Application Publication
(10) Pub. No.: US 2022/0387702 A1
(43) Pub. Date: Dec. 8, 2022

(54) DRUG DELIVERY DEVICE

(71) Applicant: AMGEN INC., Thousand Oaks, CA (US)

(72) Inventors: Scott R. Gibson, Thousand Oaks, CA (US); Mehran Mojarrad, Thousand Oaks, CA (US); Paul D. Faucher, Escondido, CA (US)

(21) Appl. No.: 17/770,331

(22) PCT Filed: Oct. 23, 2020
(86) PCT No.: PCT/US20/56987

§ 371 Date: Apr. 20, 2022

Related U.S. Application Data
(60) Provisional application No. 62/925,565, filed on Oct. 24, 2019.

(51) Int. Cl. A61M 5/142; A61M 5/168

(52) U.S. Cl. CPC A61M 5/142; A61M 5/14248; A61M 5/16854; A61M 5/14232

(57) ABSTRACT

A drug delivery device is provided that includes a housing, a fluid displacement assembly at least partially supported by and/or surrounded by the housing, and a drive component at least partially supported by and/or surrounded by the housing. The fluid displacement assembly includes a ring tube portion. The drive component includes an eccentric component having a contact surface configured to directly or indirectly apply a compression force to a compression patch of the ring tube portion such that when the eccentric component rotates about an axis, the contact surface moves along generally circular path and drives the medicament through the fluid displacement assembly. The compression force between the contact surface and the ring tube portion is preferably substantially constant throughout a complete revolution about the axis by the eccentric component.

### CROSS-REFERENCE TO RELATED APPLICATION

The present application claims priority to U.S. Application No. 62/925,565, filed Oct. 24, 2019. The priority application is hereby incorporated by reference in its entirety.

### FIELD OF DISCLOSURE

The present disclosure generally relates to drug delivery devices and, more particularly, to a pump and a system for long-term, continuous, or semi-continuous intravenous drug delivery.

### BACKGROUND

Drugs are administered to treat a variety of conditions and diseases. Intravenous (“IV”) therapy is a drug dosing process that delivers drugs directly into a patient's vein using an infusion contained in a delivery container such as IV bag and tubing connected to a needle subsystem that fluidically communicates with a reservoir through the pump assembly collectively called an infusion set. These drug dosings may be performed in a healthcare facility, or in some instances, at remote locations such as a patient's home. In certain applications, a drug delivery process may last for an extended period of time (e.g., for one hour or longer) or may include continuous or semi-continuous delivery of a drug over an extended period of time (e.g., for several hours, days, weeks, or longer). For many of these relatively long-term delivery requirements, a pump is often utilized to control and/or administer the drug to the patient. The pump may be coupled (physically, fluidly, and/or otherwise) to various components, such as a drug delivery container, supply lines, connection ports, and/or the patient.

It may be desirable to utilize a pump and/or overall system that is portable and/or wearable. It may also be desirable to utilize a pump and an overall system that minimizes patient inconvenience, minimizes the size and profile of the device and the overall system, minimizes the complexity of the device and overall system, minimizes the noise and vibration of the device, accommodates easy connection/disconnection and changeover of the infusion set, simplifies or automates priming of the line, accommodates easy delivery interruption and reestablishment based on required therapy and delivery profile, easily provides status of delivery and other important user information such as occlusion and volume of drug delivered or remaining in the reservoir, reduces the cost of the device and the overall system, increases the reliability and accuracy of the device and the overall system.

It may also be desirable to utilize a pump and/or overall system that, when activated and in a drug delivery mode, consistently and continuously delivers a relatively constant supply of medicament to the patient. It may also be desirable to utilize a pump and/or overall system that operates efficiently while minimizing power input requirements.

As described in more detail below, the present disclosure sets forth devices, systems, and methods for drug delivery embodying advantageous alternatives to existing devices, systems, and methods, and that may address one or more of the challenges or needs mentioned herein, as well as provide other benefits and advantages.

### SUMMARY

In a first embodiment, a drug delivery device is provided, including a housing, a fluid displacement assembly at least partially supported by and/or surrounded by the housing, and a drive component at least partially supported by and/or surrounded by the housing. The fluid displacement assembly includes a ring tube portion. The drive component includes an eccentric component or housing having a contact surface configured to directly or indirectly apply a compression force to a compression patch of the ring tube portion such that when the eccentric component rotates about an axis, the contact surface moves along generally circular path and drives the medicament through the fluid displacement assembly. The compression force between the contact surface and the ring tube portion is preferably substantially constant throughout a complete revolution about the axis by the eccentric component.

In some examples, the ring tube portion may define a generally circular shape. Further, in some examples, the ring tube may have a first point that overlaps with a second point. In some forms, the ring tube may have a generally spiral shape. The compression force between the contact surface and the ring tube portion may be substantially uninterrupted throughout a complete revolution about the axis by the eccentric component.

In some examples, at least a portion of the fluid displacement assembly is at least partially disposed within a disposable housing portion of the housing. In these and other examples, at least a portion of the drive component is at least partially disposed within a durable housing portion of the housing.

Further, in some approaches, the fluid displacement assembly includes a sleeve bearing and a pump race, the ring tube portion adapted to be at least partially disposed within the pump race, and to wrap around an outer periphery of the sleeve bearing. The sleeve bearing may be positioned between the eccentric component and the ring tube portion.

In accordance with a second aspect, the drug delivery device embodiments may be utilized in a drug delivery system having a drug product container containing a medicament, a fluid path configured to receive the medicament from the drug product container, and the drug delivery device positioned along and/or adjacent to the fluid path. Other examples are possible.

### BRIEF DESCRIPTION OF THE DRAWINGS

The above needs are at least partially met through provision of the systems and approaches for drug delivery device reconstitution described in the following detailed description, particularly when studied in conjunction with the drawings, wherein:

FIG. 1 illustrates an example drug delivery device in accordance with various embodiments;

FIG. 2 illustrates a partial cross-section of an example drug delivery device in accordance with various embodiments;

FIG. 3 illustrates an exploded view of an example drug delivery device in accordance with various embodiments;

FIG. 4 illustrates an exploded view of an example drive assembly for a drug delivery device in accordance with various embodiments;

FIG. 5 illustrates an exploded view of an example pump head for a drug delivery device in accordance with various embodiments;

FIG. 6 illustrates an example drug delivery device in accordance with various embodiments;

FIGS. 7-8 each illustrates an example illustration of interaction between an eccentric roller of a drive assembly and a ring tube of a fluid displacement assembly in accordance with various embodiments;

FIG. 9 illustrates a conventional drug delivery device;

FIG. 10 illustrates an alternative example drug delivery device in accordance with various embodiments; and

FIG. 11 illustrates an alternative example drug delivery device in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

The present disclosure relates to a drug delivery device and related components, such as a pump, for long-term, continuous, semi-continuous, and/or intravenous drug delivery. Under some conditions, a drug delivery process may last for an extended period of time (e.g., for one hour or longer) or may include continuous or semi-continuous delivery of a drug over an extended period of time (e.g., for several hours, days, weeks, or longer) and may include delivery via an intravenous connection to a patient. The present disclosure utilizes various features to assist with reducing noise, limiting vibration, and improving durability and overall reliability while maintaining a relatively compact sized system that may be desirable or appropriate for extended, continuous, or semi-continuous intravenous delivery.

Further, the present disclosure describes an electromechancial mechanism that may be able to deliver prescribed quantity of liquid medication from a flexible bag containing a drug or medicament. For example, the mechanism may be utilized for either self-administration or in-clinic use. The pump may have the flexibility of a removable pump-head for ease of disposal or to assist with motor assembly removal to selectively stop the fluid flow to interrupt the flow as desired per therapy requirement. The designs described herein use an efficient pump head that houses an eccentric component such as a rotor or hub in the shape of an ellipse, an inverted triangular shape, or other asymmetric shapes for delivering a desired volume of fluid per cycle of rotation. Additionally, when stopped, the pump head restricts the flow from either directions to minimize or prevent backflow or forward flow due to gravity or change in position of the components.

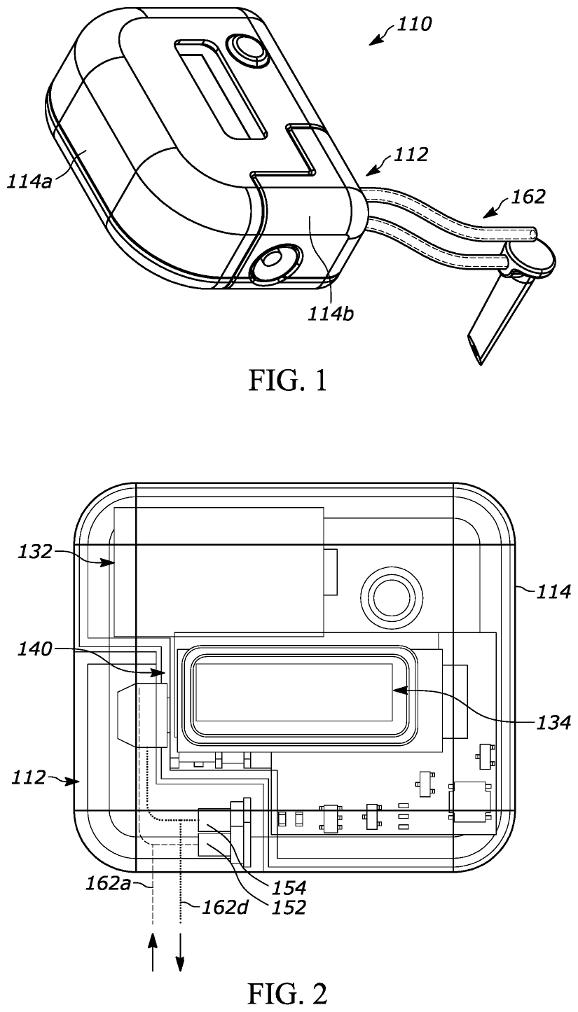

Turning to the figures, FIGS. 1 and 2 show a drug delivery device such as a pump 110 having, generally, a pump head 112 having a durable or reusable housing 114a, a disposable housing 114b, a fluid flow path 162, a power source such as a battery 132, a drive assembly such as a motor 140, a controller and display 134, and a pair of pressure sensors (e.g., inlet pressure transducer 152 and outlet pressure transducer 154). The two housing components 114a, 114b cooperate to define the overall housing 114. Additionally, in some examples, the durable housing 114a may preferably be reusable and/or durable and may be disposable as suitable. Similarly, in some examples, the disposable housing 114b may be reusable, although certain sterilization and/or refurbishment steps may be required or desirable to achieve this reusability.

As is further illustrated in FIG. 2, a medicament from a drug product container may travel through an input tube, into the pump head 112, and out of the pump through an output tube. In other words, the pump is able to urge the medicament through the pump head 112. While the pump shown in FIG. 2 is a peristaltic pump, other suitable configurations may be used, such as a positive displacement pump. The pump head 112 shown in FIGS. 1 and 2 is a ring pump that utilizes a generally circular-shaped loop of tubing 162 to create peristaltic forces. As a more specific example, the pump head 112 has a component that pinches or otherwise occludes the ring-shaped tube section in a circular motion to urge fluid through the tube 162.

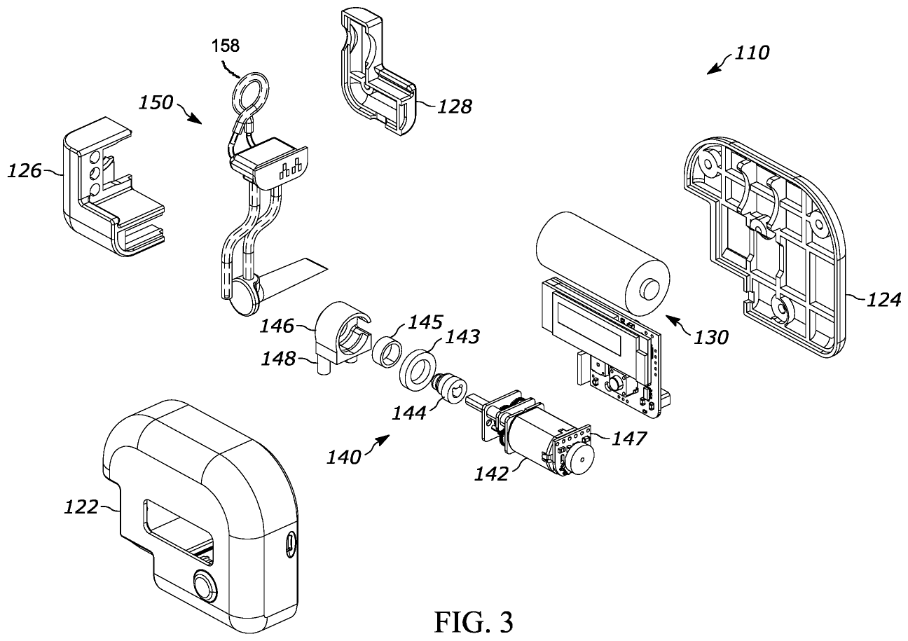

FIG. 3 illustrates an exploded view of the pump 110, including sub components of the housing 114, such as a controller front case 122, a controller rear case 124, a pump head front case 126, and a pump head rear case 128. These four components 122, 124, 126, 128 generally fit together to form at least the majority of the housing 114. These four components 122, 124, 126, 128 may be made of a generally rigid and lightweight material, such as plastic, a composite, or any other suitable material. The front/rear paired components (122, 124 on one hand, and 126, 128 on the other) may fit together via fasteners, snap-fit connections, an adhesive, or any other suitable coupling components/methods. A PCA and battery assembly 130 is at least partially contained within the housing 114, with a display screen 134 (FIG. 2) defining a portion of the housing 114.

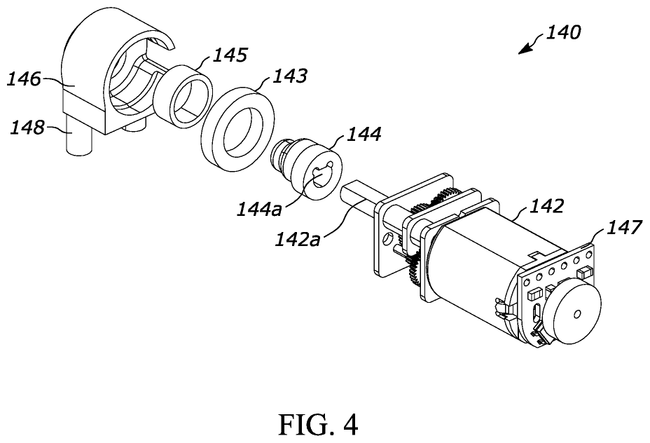

FIG. 3 further shows an exploded view of the drive assembly 140 (e.g., the motor assembly), a tube set, and pressure sensors 150. With reference to FIGS. 3 and 4, the drive assembly 140 generally includes a motor 142, a retainer ring 143, an eccentric hub 144, a sleeve bearing 145, a pump race 146, an encoder board 147, and a generally pliant/flexible isolation mount or mounts 148. The motor 142 provides a rotational driving force. The retainer ring 143 retains other components in the housing (namely the tubes, as discussed more below) and/or for aligning the eccentric hub 144. The eccentric hub 144 utilizes a cam feature to generate peristalsis. The sleeve bearing 145 provides a barrier between the eccentric hub 144 and the tubing (such as the ring tube 158). The pump race 146 is adapted to house the previously-described circular shaped tube section. The encoder board 147 is configured to measure an actual speed of the motor for increased accuracy and precision. The generally pliant/flexible isolation mounts 148 prevent part misalignment, reduce drive torque/power, and provide compliance for head installation.

As illustrated in FIGS. 3 and 4, the isolation mounts 148 allow compliance to the pump head 112. The isolation mounts 148 may be made of rubber or any other suitable material. The eccentric hub 144 includes a key portion 144a that receives a correspondingly shaped drive shaft 142a. Additionally, as shown in FIGS. 3 and 4, the eccentric hub 144, the drive shaft 142a, the motor 142, and the encoder board 147 are disposed within the durable housing 114a of the pump 112, whereas the retainer ring 143, the sleeve bearing 145, and the pump race 146 are disposed within the disposable housing 114b or the removable pump head 112. When the pump head 112 is coupled with the durable portion of the pump 110, the eccentric hub 144 aligns with and is received within the retainer ring 143. During operation, as the drive shaft 142a of the motor rotates, the eccentric hub 144 rotates on axis with the drive shaft axis 142a, and an eccentric feature produces a cyclical, annular, outward force radially onto an inner face of the circular-shaped tube section positioned within the pump race 146. More specifically, the retainer ring 143 fits around the circumference of the eccentric hub 144 to retain the ring tube 158 and the sleeve bearing 145 to prevent them from inadvertently falling out when attaching and/or detaching the pump head 112. As the eccentric hub 144 rotates, it may cause the sleeve bearing 145 to undulate and press on a relatively discrete portion of the circular-shaped tube section, thereby compressing and/or occluding that section of the tube. As the eccentric hub 144 (and the sleeve bearing 145) rotate further, the portion of the outer surface of the sleeve bearing 145 that is compressing the tube “rolls” around the inside of the pump race 146 and urges fluid in the tube to travel away from the pump head 112.

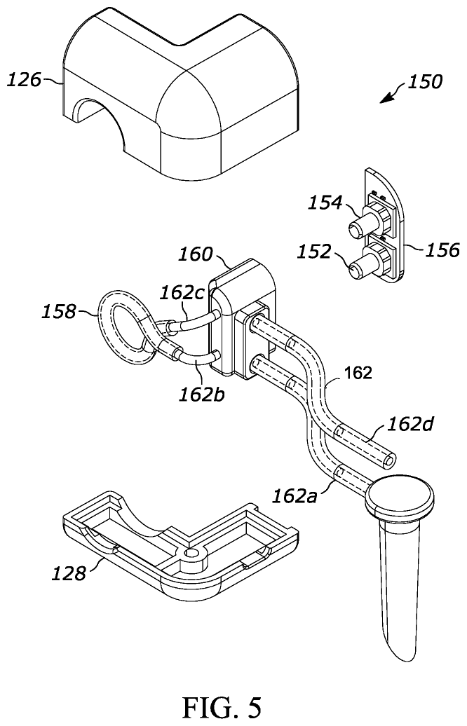

FIG. 5 shows the tube set and pressure sensors 150 in more detail, namely an exploded and enlarged view. FIG. 5 illustrates two sensors, namely inlet pressure transducer 152 and outlet pressure transducer 154, which measure fluid pressure in inlet and outlet portions of the flow path 162. The respective transducers 152, 154 shown in the figures make contact with the flow in a manifold 160 of the pump head 112. The tubing may be bonded to the manifold 160. As a more specific example, the transducers 152, 154 are electrically connected to the pump controller via sprung connector contacts and directly measure the pressure in the flow at the inlet and outlet locations 162a, 162d. As an even more specific example, each transducer 152, 154 is electrically connected to a pressure transducer board 156 that is electrically connected to other electronic controls such as a motherboard. For example, the transducers 152, 154 shown in the figures are each mounted on the pressure transducer board 156.

Each transducer 152, 154 shown in the figures may include a diaphragm, made from the same or similar material as the tubing, placed inline on both the inlet and outlet tubes 162a, 162d. These diaphragms are located in the pump head 112 and make contact with a portion of the pump controller (e.g., the pressure transducer board) when the pump head assembly is installed via the pressure transducer board 156. At the point of diaphragm contact, load cells in the pump controller monitor variation in force exerted by the diaphragm which correlates to pressure changes in the flow. In this manner, the flow rate can be monitored at the inlet and outlet of the pump head 112 to provide the pressure sensor benefits discussed herein while not needing to introduce new materials into contact with the drug. It will be appreciated that other or alternative types of pressure sensors may be utilized, such as non-contact pressure sensors design to provide the benefits of pressure sensors but without the risk of material non-compatibility.

In flow systems having a rigid fluid path, monitoring the speed of the pump head may be all that is necessary for precise flow control with a positive displacement pump using the following equation: flow rate=[volume/revolution]*[revolutions/time]. However, in IV-based fluid systems, it may be beneficial to use a fluid path constructed from flexible tubing that expands and contracts with pressure, which subsequently affects the volume of product in peristaltic systems and may decrease effective accuracy. This pressure variation can occur from the variation in height of the IV bag with respect to the controller and/or pump, as well as from partial occlusion or other environmental influences. As a result, the effectiveness of flow control may depend on assumptions of fluid input pressure.

FIG. 5 also shows an example of the fluid flow path 162 in more detail. For example, the fluid flow path 162 may include an external tubing inlet side portion 162a, an internal tubing inlet side portion 162b, an internal tubing outlet side portion 162c, and an external tubing outlet side portion 162d. The various portions of tubing 162a-d may be integrally formed (i.e. a single piece of tubing), or they may be made of two or more sections of tubing that are fluidly connected with each other. The external tubing portions 162a, 162d shown in the figures may be constructed from the same type and sized tubing and may be the same type and size of tubing used in IV lines. In some examples, the internal tubing portions 162b, 162c may each be constructed from a smaller diameter tube to facilitate pressure measurement. The flow path 162 may also include a fluid displacement assembly, such as a ring tube 158, i.e., the previously-discussed generally circular portion of tubing that is housed within the pump race 146. In one embodiment, the ring tube 158 defines the boundary between the inlet fluid flow path and the outlet fluid flow path. As previously noted, the pump head 112 components depicted in FIG. 5 are supported by the pump head front and rear case 126, 128 and the pump head 112 is removably coupled with the remainder pump structure. The pump head 112 may be disposable and the remainder pump structure may be reusable (e.g. “durable”).

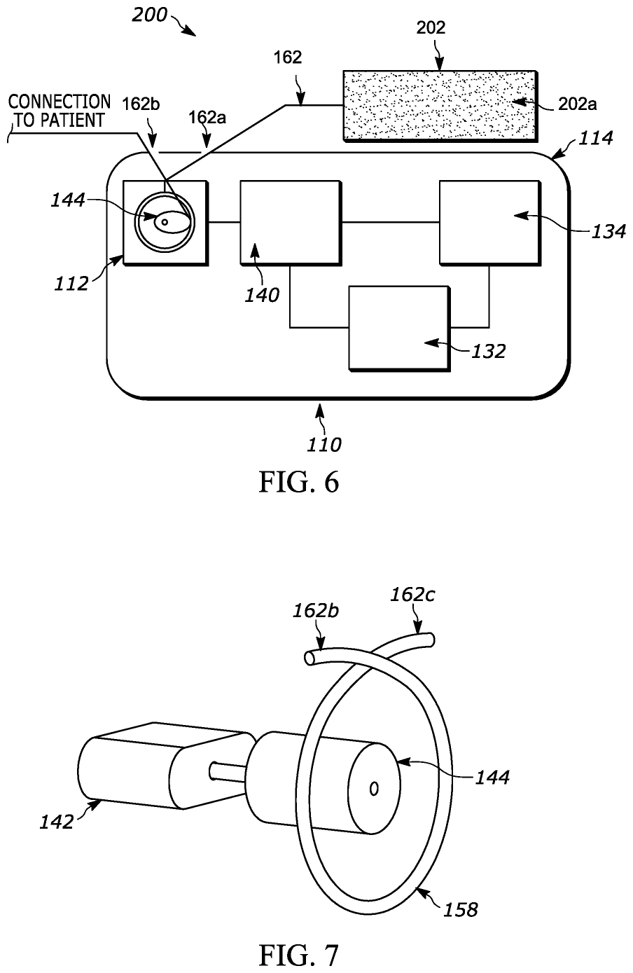

FIG. 6 shows an example drug delivery system 200 illustrating a drug product container (e.g., a reservoir 202) containing a medicament 202a, a fluid flow path (e.g., tubing 162) connecting the drug product container to a pump and then to a patient, and a drug delivery device (e.g., a pump having a housing). The drug delivery device 110 shown in FIG. 6 includes a controller and display 134 , a battery 132, a motor assembly 140, and a pump head 112, each of which is substantially or completely contained within and/or supported by the housing 114.

FIG. 7 shows an isometric view of a spiral-shaped ring tube 158 in a generally circular shape positioned around an eccentric component (e.g. roller 144). The pump housing/ring housing are not shown for illustrative purposes.

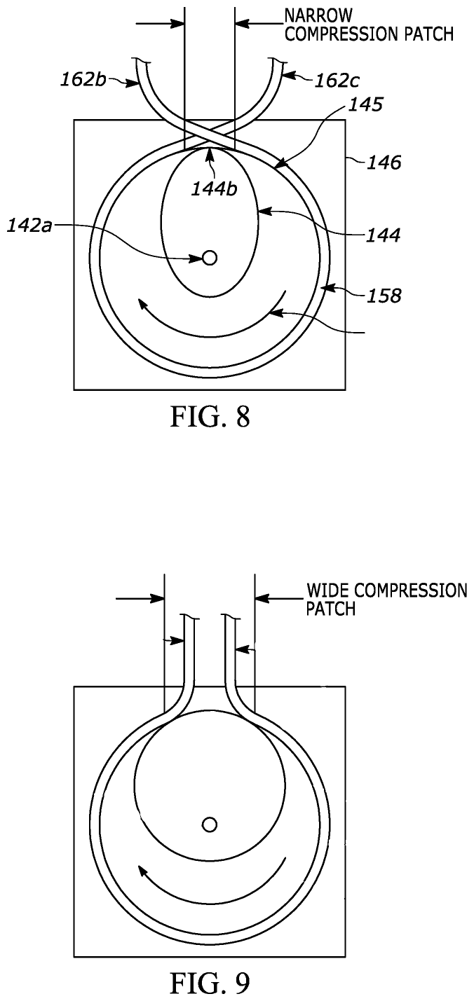

FIG. 8 shows the spiral-shaped ring tube 158 from FIG. 7 disposed within the pump housing/ring housing. As shown in the figure, the eccentric roller 144 has a discrete point (i.e. contact surface 144b) that contacts the tubing to form a fluid-tight or substantially fluid-tight seal at that point of the tube ring 158. Then, as the eccentric roller 144 rotates, the fluid positioned upstream (clockwise in FIG. 8) of the tube ring is then urged forward towards the patient (i.e., towards the outlet 162c), while also pulling fluid from the IV bag on the backside of the roller.

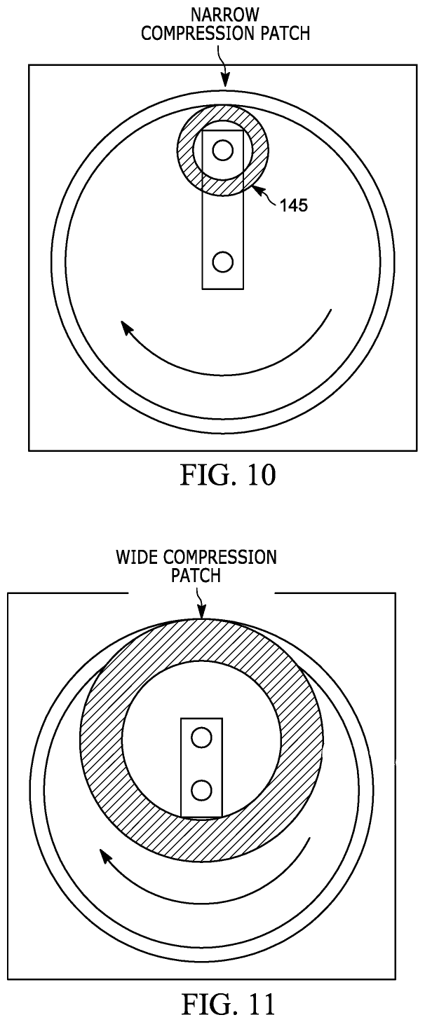

Because the inlet and outlet portions 162b, 162c of the tube ring 158 overlap or cross each other, the device is able to have a relatively narrow contact surface while maintaining the fluid-tight seal with both the inlet and outlet portions of the tube ring 158, even when the eccentric roller contact surface 144b stops while in-line with the area where the inlet and outlet overlap. Because the eccentric roller has a narrow contact surface, the tube ring 158 also has a narrow compression patch (area that is compressed, see FIG. 10) while still maintaining a suitable seal at all times.

In one configuration, the compression patch is less than 6 mm wide, while still maintaining a suitable seal at all times. In another configuration, the compression patch is less than 5 mm wide while still maintaining a suitable seal at all times. In another configuration, the compression patch is less than 4 mm wide while still maintaining a suitable seal at all times. In another configuration, the compression patch is less than 3 mm wide while still maintaining a suitable seal at all times. In another configuration, the compression patch is less than 2 mm wide while still maintaining a suitable seal at all times. In another configuration, the compression patch is less than 1 mm wide while still maintaining a suitable seal at all times.

Advantageously, the spiral ring pump configuration offers a relatively low energy (high efficiency/low power consumption) fluid drive mechanism for a drug delivery device. The spiral ring pump fluid drive mechanism provides multiple advantages compared to existing designs, including requiring less energy per revolution than conventional peristaltic or ring pump designs for a given fluid tube size, increasing efficiency and reducing power consumption. Further, the system described herein may deliver an increased quantity of fluid per revolution than conventional peristaltic or ring pump designs for a given fluid tube size. This may reduce the number of pump revolutions needed to dispense a given aliquot or dose size, increasing efficiency and reducing power consumption. The system may be configured to deliver high flow rates, which minimize the duration of “active” periods (pump energized) for delivery of each aliquot, increasing efficiency and reducing power consumption. In some examples, the pump controller and firmware may be configured to enter a low-power “sleep” state (pump de-energized and controller in a minimum power state) between active periods when drug is dispensed, increasing efficiency and reducing power consumption. When the pump is de-energized, the spiral ring pump mechanism may substantially or completely seal the fluid path at any desired stopping position. This reduces or eliminates the need to restrict or control the position at which the pump stops when de-energized. Because the fluid path is sealed whenever the pump is de-energized, both backflow of bodily fluids (flow into the device and/or drug reservoir), and/or unwanted forward flow of drug into the patient caused by external forces (e.g., head pressure caused by elevating the IV bag above the patient or compression of the IV bag) are reduced or prevented. This increases dosage accuracy and reduces the likelihood of clots in the device fluid path.

The potential reduced power consumption of the spiral ring pump fluid drive mechanism provides multiple benefits, especially when used in portable/wearable device applications, including, for a given battery size, as power consumption decreases, device runtime may increase. For a given runtime, as power consumption decreases, battery size (and thus overall device size) decreases. As battery and/or device size decrease, patient comfort and/or device usability generally increase. Further, as battery and/or device size decrease, device cost generally decreases.

The fluid path tubing (IV line) may be initially separate from the flexible reservoir (IV bag). The tubing is attached to the flexible reservoir, then primed and installed into the pump head in a spiral configuration where the inlet and outlet lines cross as they exit the pump ring.

The spiral ring pump mechanism creates fluid flow by peristalsis acting on the fluid path tubing. An eccentric roller has a contact surface (e.g., area where the inner walls of the tubing are compressed against the inner surface of the pump ring) forming a localized fluid seal across the inside surface(s) of the tubing. When energized, the pump motor rotates the eccentric roller, causing the contact surface to move in a circular path, which induces a net fluid flow from pump inlet to pump outlet.

In order to prevent unwanted flow across the pump head when the pump is de-energized, it is beneficial for the pump mechanism to maintain a fluid seal across the tubing at any orientation where the eccentric roller may stop.

On the other hand, in a ring pump with inlet and outlet lines that do not cross each other (e.g., FIG. 9), the inlet and outlet tubes may be parallel and co-planar as they exit the pump head. Therefore, unless a control system is used to prevent the roller from stopping at the inlet/outlet position, the compression patch must be made wide enough to simultaneously seal both the inlet and outlet tubes to ensure that a seal is maintained across both if the eccentric roller were to stop at the tubing inlet/outlet position when the pump is de-energized. Conversely, in a spiral ring pump (e.g., FIGS. 7, 8), because the inlet and outlet lines cross as they exit the pump ring, the compression patch may be made narrower than for a similar conventional ring pump while still ensuring unwanted flow is prevented when the pump is de-energized.

For a given tubing size and material, as the size of the compression patch increases, the energy consumed by a ring pump increases while efficiency decreases. Similarly, as the width of the compression patch increases, the fluid delivered per pump revolution also decreases because a smaller proportion of the tubing loop is available for fluid transport. Therefore, because the compression patch is narrower for a spiral ring pump (FIGS. 7-8) than for a conventional ring pump (FIG. 9), the energy efficiency of a spiral ring pump is higher than that of conventional ring pump (for a given tubing size and material). In other words, in all roller positions (except the tubing inlet/outlet position), the energy needed to operate a spiral ring pump may be less than that of a conventional ring pump. Additionally, because fewer pump revolutions are needed to deliver a given volume of fluid, the energy needed to operate a spiral ring pump may be less than that of a conventional ring pump.

In typical applications where aliquots are delivered periodically to the patient (e.g. hourly) over an extended period (days/weeks/months), the spiral ring pump may be configured to be have a sufficiently high flow rate such that the time required to deliver each aliquot is relatively short (e.g. 1 minute). By minimizing the time that the spiral ring pump is energized, the total energy consumed by the device may be reduced by configuring the controller to enter a low energy “sleep” state during the periods between aliquot delivery. Therefore, as the pump duty cycle (the ratio of pump on-time to off-time) decreases, the proportion of time that the controller is in a low energy sleep state increases, and thus total energy consumed by the device decreases.

In typical IV pump applications where aliquots are delivered periodically to the patient (e.g. hourly) over an extended period (days/weeks/months), it is important that unintended fluid flow through the device is prevented. This includes backward flow of bodily fluids into the pump and/or reservoir, which may lead to clots or clogs in the fluid path, as well as undesired forward flow of drug into the patient. As described above, the spiral ring pump prevents unintended fluid flow through the device by maintaining a fluid seal across the tubing within the pump head at all angular positions of the eccentric roller.

The various components, devices, embodiments, and systems described may be advantageous over known components, devices, and systems for a number of reasons. For example, the pump designs and/or embodiments disclosed herein have a reduced, size, weight, and overall footprint compared to known pump designs. This advantage may offer dramatic quality of life and/or convenience for patients using the pump designs. As another example, the pump designs and/or embodiments disclosed herein may have an improved dose accuracy. As yet another example, the pump designs and/or embodiments disclosed herein may have a reduced complexity of the device and overall system. As yet another example, the pump designs and/or embodiments disclosed herein may have a reduced pump noise. As yet another example, the pump designs and/or embodiments disclosed herein may have a reduced cost of the device and the overall system. As yet another example, the pump designs and/or embodiments disclosed herein may have a reduced reliability of the device and overall system. As yet another example, the pump designs and/or embodiments disclosed herein may have an increased product life of the device and overall system.

In some examples, the system may be utilized with medicament in the form of a half-life extended bispecific T cell engager (BITE®). For example, the active pharmaceutical ingredient (“API”) may be between approximately 2 mcg and approximately 100 mcg, depending on the BiTE® and container size, which, may be in a powdered form (i.e., lyophilized) requiring reconstitution. In other examples, the drug product may be in liquid form and may not require reconstitution. Nonetheless, the system includes an accurate quantity of drug product, and thus does not require the need to add additional quantities thereto in a sterile environment. In some examples, the API may be in the form of a half-life extended (“HLE”) BiTE® and/or an IV-admin monoclonal antibody (“mAbs) as desired. These HLE BiTE®s include an antibody Fc region that advantageously provides different drug properties such as longer and extended half-lives. Accordingly, such APIs may be preferred due to their ability to maintain protective levels in the patient for relatively longer periods of time. Nonetheless, in other examples, the API may be in the form of a canonical-BITE® that is to be administered in a professional healthcare environment.

The drug product container may be in the form of an IV bag, a vial, a prefilled syringe, or similar container that includes a reconstitution container body defining an inner volume. The inner volume may be sterile. In some approaches, the reconstitution container adapter may also be a CSTD (or, in examples where the prefilled reconstitution container is in the form of a syringe, the container adapter may be a needle) that mates, engages, and/or couples to the vial adapter. Additionally or alternatively, the drug product can be bulk lyophilized and filled into a cartridge or container that is typically used to administer with an IV pump. If needed the dehydrated forms of IVSS, NaCl, and any other components needed for the final administered solution can be bulk lyo'ed and filled into the cassette for long term storage.

The system may be distributed and/or sold as a common kit packaging, but other suitable distribution/packaging is suitable. The drug product may be in the form of a half-life extended bispecific T cell engager (BITE®), but other drug products are suitable. The diluent include water for injection (“WFI”), but other diluents may be suitable. The containers may be pliable bags, such as IV bags, but other containers may be suitable. In some examples, one or more of the containers is in the form of an IV drip bag constructed from a plastic or other material, e.g., 250 mL 0.9% Sodium Chloride IV bag constructed of a suitable material such as polyolefin, non-DEHP (diethylhexl phthalate), PVC, polyurethane, or EVA (ethylene vinyl acetate) and can be filled to a volume of approximately 270 mL to account for potential moisture loss over long-term storage.

In some examples, the prefilled delivery container is in the form of an IV drip bag constructed from a plastic or other material, e.g., 250 mL 0.9% Sodium Chloride IV bag constructed of a suitable material such as polyolefin, non-DEHP (diethylhexl phthalate), PVC, polyurethane, or EVA (ethylene vinyl acetate) and can be filled to a volume of approximately 270 mL to account for potential moisture loss over long-term storage. Other examples of suitable delivery containers are possible such as, for example, a glass bottle or container. Example suitable prefilled delivery containers are described in U.S. Appln. No. 62/804,447, filed on Feb. 12, 2019 and U.S. Appln. No. 62/877,286 filed on Jul. 22, 2019, the contents of each of which are incorporated by reference in their entirety.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like;Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Solids™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP Ilb/Ilia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF α monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)-N-((S)-1-((S)-4-methyl-1-((R)-2-methyloxiran-2-yl)-1-oxopentan-2-ylcarbamoyl)-2-phenylethyl)-2-((S)-2-(2- morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA) x anti-CD3 BiTE® (bispecific T cell engager) construct. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BITE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1(PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device for delivering a medicament, comprising:
a housing;
a fluid displacement assembly at least partially supported by and/or surrounded by the housing, the fluid displacement assembly including a ring tube portion;
a drive component at least partially supported by and/or surrounded by the housing, the drive component including an eccentric component having a contact surface configured to directly or indirectly apply a compression force to a compression patch of the ring tube portion such that when the eccentric component rotates about an axis, the contact surface moves along generally circular path and drives the medicament through the fluid displacement assembly;
wherein the compression force between the contact surface and the ring tube portion is substantially constant throughout a complete revolution about the axis by the eccentric component.

2. The drug delivery device as in claim 1, wherein the ring tube portion defines a generally circular shape.

3. The drug delivery device as in claim 1, wherein the ring tube portion includes a first point that overlaps with a second point.

4. The drug delivery device as in claim 1, wherein the ring tube defines a fluid flow path having a generally spiral shape.

5. The drug delivery device as in claim 1, wherein the compression force between the contact surface and the ring tube portion is substantially uninterrupted throughout a complete revolution about the axis by the eccentric component.

6. The drug delivery device as in claim 1, wherein at least a portion of the fluid displacement assembly is at least partially disposed within a disposable housing portion of the housing.

7. The drug delivery device as in claim 1, wherein at least a portion of the drive component is at least partially disposed within a durable housing portion of the housing.

8. The drug delivery device as in claim 1, wherein the fluid displacement assembly includes a sleeve bearing and a pump race, the ring tube portion adapted to be at least partially disposed within the pump race, and to wrap around an outer periphery of the sleeve bearing.

9. The drug delivery device as in claim 8, wherein the sleeve bearing is positioned between the eccentric component and the ring tube portion.

10. A drug delivery system for delivering a drug product, comprising:
a drug product container containing a drug product;
a fluid path configured to receive the drug product from the drug product container; and
a drug delivery device positioned along and/or adjacent to the fluid path;
wherein the drug product container includes:
a housing;
a fluid displacement assembly at least partially supported by and/or surrounded by the housing, the fluid displacement assembly including a ring tube portion;
a drive component at least partially supported by and/or surrounded by the housing, the drive component including an eccentric component having a contact surface configured to directly or indirectly apply a compression force to a compression patch of the ring tube portion such that when the eccentric component rotates about an axis, the contact surface moves along generally circular path and drives the medicament through the fluid displacement assembly;
wherein the compression force between the contact surface and the ring tube portion is substantially constant throughout a complete revolution about the axis by the eccentric component.

11. The drug delivery system as in claim 10, wherein the ring tube portion defines a generally circular shape.

12. The drug delivery system as in claim 10, wherein the ring tube portion includes a first point that overlaps with a second point.

13. The drug delivery system as in claim 10, wherein the ring tube defines a fluid flow path having a generally spiral shape.

14. The drug delivery system as in claim 10, wherein the compression force between the contact surface and the ring tube portion is substantially uninterrupted throughout a complete revolution about the axis by the eccentric component.

15. The drug delivery system as in claim 10, wherein at least a portion of the fluid displacement assembly is at least partially disposed within a disposable housing portion of the housing.

16. The drug delivery system as in claim 10, wherein at least a portion of the drive component is at least partially disposed within a durable housing portion of the housing.

17. The drug delivery system as in claim 10, wherein the fluid displacement assembly includes a sleeve bearing and a pump race, the ring tube portion adapted to be at least partially disposed within the pump race, and to wrap around an outer periphery of the sleeve bearing.

18. The drug delivery system as in claim 17, wherein the sleeve bearing is positioned between the eccentric component and the ring tube portion.
